(19) United States
(12) Patent Application Publication
(10) Pub. No.: US 2009/0220053 A1
(43) Pub. Date: Sep. 3, 2009

(54) Sensor protection systems and methods

(76) Inventors: Riccardo J. Tresso, Westminster, CO (US); Michael J. Vega, Longmont, CO (US)

Correspondence Address: LAW OFFICE OF CHAD C. SOLIZ , LLC, 407 N. Lincoln Ave. Ste. 105, LOVELAND, CO, 80537, US

(21) Appl. No.: 12/380,855

(22) Filed: Mar. 3, 2009

Related U.S. Application Data
(60) Provisional application No. 61/068,284, filed on Mar. 3, 2008.

(51) Int. Cl. A61B 6/14; H01J 31/50

(52) U.S. Cl. 378/168; 378/189

(57) ABSTRACT

An intra-oral sensor protection device in accordance with the present invention facilitates the comfort of the patient during radiology examination, while protecting sensitive and relatively delicate digital sensors from damage during the examination. Features of the present invention address protection of the sensor during intra-oral manipulation and placement of the sensor during examination, providing a fluid barrier, and variable positioning features, all while facilitating the ease of use of the sensor in radiographic examination in preparation for, during and after the exam. The intra-oral sensor protection device has two sensor protection elements that are releasably retained in either open or closed configurations to provide a protective casing for a sensor. Features are provided to accommodate for either wired or wireless sensor communication and may have sensor protection connection elements that provide for a fluid barrier when each sensor protection element is retained in a closed configuration. A sensor may be releasably retained within an interior of the two sensor protection elements, the sensor in preferred embodiments being retained within the two sensor protection elements. The present invention relates and is directed to apparatus and methods of dentistry and dental examination. The present invention also provides processes and methods directed to one or more aspects of intra-oral radiological dental examination.

### BACKGROUND OF THE INVENTION

The present invention relates to systems for the protection of sensors used in dental examination, and specifically, the protection of intra-oral sensors such as traditional digital sensors used in radiograph dental examination techniques, both wired and wireless technologies, and in some particular embodiments, to apparatus and methods of dentistry and dental examination. The present invention may also provide processes and methods directed to one or more aspects of dentistry and dental examination, such as to accommodate intra-oral sensors, providing one or more of: protection of patient and equipment, fluid barrier, patient barrier, comfort, ease of use, reliable fluid barrier, and variable bite tab locations, as well as the use of digital sensors and other examination equipment.

Reference to a patient is made throughout this specification. The reference may be made to human patients, for example, or alternatively reference can be made to a designation of an animal, such as in veterinary practice, or in testing environments upon live subjects, test equipment, and forensics, as but a few of the possible applications. In any event, each any examined subject may be referred to generally as a patient and the present invention may be applicable to the above applications and in the examination and care thereof as one of ordinary skill in the art would understand such technology.

Reference to sensors, intra-oral sensors, digital radiograph sensors or other examination sensors is made throughout this specification. The reference may sometimes be made to other intra-oral sensor examinations and dental examinations now known or later developed, for example any intra-oral examination sensor technique, or other reference to a dental examination practice, or in testing environments upon live subjects, test equipment, or otherwise. In any event, each may be referred to generally as examination that is intra-oral or otherwise conducted within the oral cavity and the present invention is applicable to the above applications, including but not limited to sensor technologies incorporating wired or wireless technologies, as one of ordinary skill in the art would understand such technology.

Managing examination in dentistry environments can be a daunting undertaking that may require specialization and experience in not only dentistry but in various fields of dental assisting, hygiene, and examination processes, scanning science, data acquisition and processing, as well as business practices. The ultimate objective may be the protection and adequate examination of the patient to achieve an adequate examination and preferred care. However, the accommodation of digital radiograph or other sensors that may be used in traditional practice must be considered, while also providing for the reliable protection of this and other examination equipment, such as providing a fluid barrier for the equipment in light of the type of examination and technique as well as protection from external forces that may damage sensitive equipment and sensors. Other factors must also be considered, such as the comfort of the patient, ease and efficient of use of examination equipment, and variable bite tab locations that may be required in traditional examination techniques. The everyday business operations and revenue of practice may in indeed be impacted by the type of equipment and methods chosen to accomplish effective examination practices.

Systems have been devised in the industry in an attempt to accommodate for both the traditional examination of patients utilizing typically digital sensors for digital radiograph examination of the patient while providing for some basic level of functionality in protecting the sensor used and attempting to provide for an economical solution for everyday operations. The traditional technology may provide a combination of fluid barrier and positional alignment via vinyl bag, some portion of plastic cover, and general use sensor holders with varying packaging profiles that may also provide data cable strain relief features for wired applications. These technologies may require further accessories apart from any protector or cover and may not provide adequate protection of the intra-oral sensor given the need also for cost-effective equipment that is easy to use in practical examination practice. These traditional technologies may further be restricted to traditional RINN system positioning of the protector or cover and bite tab or bite wing. Other art provides basic hinged enclosure and fixed bite tab that may require external positioning devices. The concept of enclosures, covers or protectors for digital sensors, film and phosphor plate, as well as fixed bite tab technologies, may be generally known and reflected in traditional technologies, technologies that may presently lack some or many of the above-mentioned dental examination and business practice objectives.

Traditional technologies may suggest that there has been a heretofore identified but yet unanswered need for an inclusive intra-oral examination solution to achieve the above-stated objectives while providing for the everyday business operations and economic value necessary in order to maintain the traditional intra-oral examination as a viable business and medical practice. Heretofore known dentistry services, for example, may provide for and perform the function of intra-oral examination well, including the provision of some functionality in a digital sensor examination solution, while leaving many of the objectives achieved by the present invention lacking or unaddressed.

Traditional digital sensor technologies may consist of a CCD array with additional structural features for strain relief of the wired data cable or streamlined, self contained wireless transmitters, each usually varying the difference between the commercial products only by the packaging profile and data cable strain relief geometry.

It may be that further identified, but as yet unaddressed, needs of industry reflect a recognition that “one size fits all” approaches for developing sensor enclosures are not practical given the profile and thickness variations in the industry. Furthermore, a developed enclosure covering, say, in standard sizes 0, 1, 2, 3 and 4 products, may be too bulky and impractical.

Heretofore known technologies may be directed to intra-oral examination using digital sensors and enclosure, cover or protector technologies; however, as far as they appear to be no current technologies that provide or even attempt to solve many or all of the above problems, fail to fully address the identified needs, and that lack the functionality and inventive features of the present invention. Many of the traditional systems require many different components, such as bite wings, foam bite tabs, foam pads to protect the sensor body, vinyl fluid barrier covers, foam envelops to protect the sensor, and positioning systems of foam or other materials, making the traditional solution cumbersome and impractical.

### SUMMARY OF THE INVENTION

The present invention relates to systems for protecting intra-oral sensors used in dental examination, such as traditional intra-oral digital radiograph sensors, both wired and wireless technologies, and in some particular embodiments, to apparatus and methods of dentistry and dental examination. The present invention also provides processes and methods directed to one or more aspects of dentistry and dental examination, such as to accommodate intra-oral sensors, providing for the protection of patient and equipment, a reliable fluid barrier, a patient barrier, comfort of the patient, ease of use, and variable positioning features, as well as the use of digital sensors and other examination equipment. The present invention is a relatively low cost item that may be provided in some embodiments as a disposable enclosure.

An intra-oral sensor protection device in accordance with the present invention facilitates the comfort of the patient during radiology examination, while protecting sensors such as sensitive and relatively delicate digital sensors from damage during the examination. Features of the present invention address protection of the sensor during intra-oral manipulation and placement of the sensor during examination, providing a fluid barrier, and variable positioning features, all while facilitating the ease of use of the sensor in radiographic examination in preparation for, during and after the exam.

The intra-oral sensor protection device in preferred embodiments has two sensor protection elements with fixed outer profiles that releasably retain a sensor and define in preferred embodiments a fixed profile sensor casing. The two sensor protection elements are releasably retained in either open or closed configurations. The two sensor protection elements may have a wired or wireless sensor communication element to accommodate for a sensor to be retained. The first and second sensor protection elements provide a fluid barrier about the extent of the sensor. In some preferred embodiments, the intra-oral sensor protection device may have sensor protection connection elements that provide for a fluid barrier, a fluid barrier facilitated when each sensor protection element is retained in a closed configuration. A sensor is releasably retained within an interior of the two sensor protection elements in the closed configuration, and in preferred embodiments the sensor is entirely contained within the two sensor protection elements. Accordingly, the fixed profile sensor casing contains the extent of the sensor within the sensor protection interior and provides a barrier about the extent of said sensor protecting the sensor and the patient.

Methods of protecting a sensor during intra-oral examination are disclosed. In some embodiments of the present invention, a method of protecting a sensor during intra-oral examination has the steps of positioning a sensor in an interior of a first sensor protection element, connecting the first sensor protection element with a second sensor protection element, releasably retaining the first and second sensor protection elements in a closed configuration, defining a sensor protection interior by the first and second sensor protection elements in the closed configuration, releasably retaining the sensor within the sensor protection interior in the closed configuration, and providing a fluid barrier with said first and second sensor protection elements about the extent of said sensor. A barrier generally may be provided between the sensor and the patient protecting both and adding comfort during the examination. Other steps may be performed in accordance with the present invention and may be described functionally herein as embodiments of the invention.

Methods of intra-oral examination are also disclosed. In some embodiments of the present invention, a method of intra-oral examination begins by providing a sensor for intra-oral examination, positioning a sensor in an interior of a first sensor protection element, connecting the first sensor protection element with a second sensor protection element, releasably retaining the first and second sensor protection elements in a closed configuration, defining a sensor protection interior by the first and second sensor protection elements in the closed configuration, releasably retaining the sensor within the sensor protection interior in the closed configuration, providing a fluid barrier with said first and second sensor protection elements about the extent of said sensor, placing the sensor in an intra-oral location of a patient for radiological dental examination, and performing dental examination of the patient. Again, other steps may be performed in accordance with the present invention and may be described functionally herein as embodiments of the invention.

In some embodiments, the invention may have sensor protection connection features such as fluid barrier connections, and in some preferred embodiments lap joint features, fluid barrier features that may even in some embodiments provide a fluid barrier the extent of a sensor communication element. Furthermore, the first and second sensor protection elements may be connected by a hinge element, the first and second sensor protection elements being releasably retained in open or closed configurations—in some preferred embodiments considered as a two piece ‘living hinge’.

As previously described, the invention may have positioning features such as positioning elements for intra-oral radiological examination, and in some embodiments, have adjustable bite tab elements that may be provided in bite wing, anterior and posterior configurations. The positioning elements may be adjustable and may also serve to releasably retain the first and second sensor protection elements. Guide elements may provide to adjustably retain the positioning elements. In some embodiments, the adjustable bite tab elements may be configured in shape and adjustable positioning based upon the intended use and examination technique. Locating elements may be used to adjustably retain the position of the positioning elements.

In still further embodiments, the first and second sensor protection elements may having a sensor communication element that in some embodiments may extend as first and second surfaces of the respective first and second sensor protection elements. The sensor communication element may not only provide location and retention of an electrical communication element such as wire or cable of the sensor, but may serve as additional protection and fluid barrier for the sensor and the cable itself. In some embodiments these features may be considered a cable relief in accordance with the invention. In alternative embodiments, a sensor communication element may accommodate wireless communication and features of the sensor, such as an antenna, transmitter, or other elements.

The intra-oral sensor protection device in some embodiments may have features that facilitate releasably retaining the first and second sensor protection elements from a closed to an open configuration, while additional elements may also be included that further facilitate releasably retaining the first and second sensor protection elements from an open to a closed configuration, and in some preferred embodiments may be interference elements or locking elements. Other additional features may be included as embodiments of the present invention, such as one or more sensor positioning elements, such as locating elements providing releasable retention of the sensor within the first and second sensor protection elements, and even in some preferred embodiments providing horizontal and top and bottom location of the sensor within the first and second sensor protection elements. Support elements within the first and second sensor protection elements may be included to support the sensor.

Design features such as the shape and configuration of the first and second sensor protection elements comprise aspects of the invention, such as rounded corners of surfaces and surface configuration of the intra-oral sensor protection device in open and closed configurations. Combinations of some or all of the above features, as elements, steps or expressed or implied functionality of the invention, may be provided to accommodate sensor and/or data cable or wireless features, providing overall improved protection over traditional technologies, while affording a reliable fluid barrier, a patient barrier, comfort, ease of use, and variable positioning element locations for flexibility during exam.

### BRIEF DESCRIPTION OF THE DRAWINGS

FIGS. 1A and 1B are isometric views of the interior and exterior of an embodiment of the present invention in an open configuration.

FIGS. 2A and 2B are isometric views of an embodiment of the present invention in a closed configuration.

FIG. 3 is an isometric view of an embodiment of the present invention and describing some positioning features of the present invention.

FIGS. 4A and 4B are isometric views of a posterior positioning feature in accordance with the present invention.

FIG. 5 is an isometric view of a Prior Art sensor.

FIG. 6A is an isometric view of another embodiment of the present invention having sensor communication protection features and described in relation to a sensor and sensor communication; FIG. 6B is a side view of the embodiment of FIG. 6A in an open configuration and described in relation to a sensor and sensor communication.

FIG. 7 is a back and partial cross-sectional view of the embodiments of FIGS. 6A and 6B in an open configuration.

FIGS. 8A and 8B are isometric views of the interior and exterior of an embodiment of the present invention in an open configuration; FIGS. 8C and 8D are isometric views of the embodiments of 8A and 8B described in relation to a sensor and sensor communication; FIG. 8E is an isometric view of the embodiments of FIGS. 8A to 8D and describing some positioning features of the present invention; FIG. 8F is a detailed view of a closing feature embodiment of the present invention.

FIGS. 9A to 9D are back and partial cross-sectional views of some embodiments of the present invention.

FIG. 10 is a flow chart describing an embodiment of the present invention.

FIG. 11 is a flow chart describing an embodiment of the present invention.

### DETAILED DESCRIPTION OF PREFERRED EMBODIMENTS

The invention in some embodiments are processes and methods, as well as one or more apparatus and device, each embodiment providing functionality through either the apparatus or device or by way of the disclosed process and methods. The invention as previously described and as further described below is technology suitable for known and to be developed technologies incorporating various sensors, such as traditional digital radiographic sensors. Those skilled in the art are familiar with traditional digital sensors for intra-oral examination.

Furthermore, aspects of the invention may be described to be composed of preferred materials, while other embodiments can be provided in other materials as alternatives encompassed by the present invention as one skilled in the art would be familiar with the disclosed and equivalent materials. In preferred embodiments of the invention, the material has pliable characteristics that facilitate the objects of the present invention, but that also may add to the workability of the technology during examination procedures and may even add to the comfort of the patient.

Embodiments of the invention may be described herein for wired sensor technology; however, the invention further provides alternatively for wireless technologies. The present invention is also applicable to disposable embodiments that are preferentially designed to be a relatively low cost solution.

Embodiments of the invention may be now or later illustrated as a diagram of the functionality of the system or even as a configuration of an intra-oral examination system as but one embodiment of the present invention. The system may comprise the invention as well as known examination features and techniques. The invention may be described as a protection device used in some preferred embodiments for intra-oral examination and will preferably fit both standard size and other size variations of sensors. Although the disclosure may specifically reference standard size zero (0) digital radiograph systems, such as the Kodak RVG 6100 system, the technology and functionality of the present invention are applicable to other sizes, such as standard sizes 0, 1, 2 3 and 4 and ‘plus’ sizes, and other systems for intra-oral sensors. Moreover, the invention may be made to accommodate any size sensor or system as understood by one skilled in the art and in accordance with the present invention. Some embodiments may accommodate sensors with a CCD thickness of less than or equal to 5 mm. A plus size may designate sensors with a CCD thickness of greater than 5 mm.

Traditional digital sensor technologies consist of CCD arrays, with additional structure features for strain relief of wire data cable, referred to generally as sensor communication, or streamlined, self contained wireless transmitter for wireless sensors. As presently there are only a few OEM manufactures using CCD technology, the difference between products may be the packaging profile and data cable strain relief geometry. Furthermore, traditional intra-oral examination may require specific positioning of the sensor in the mouth of the patient, primarily what is known as bitewing orientation, posterior, anterior and occlusal. The present invention and its features seek to accommodate the traditional sensor technologies and examination techniques in a novel approach to protecting the sensor and in intra-oral examination, as well as in the inventive devices herein.

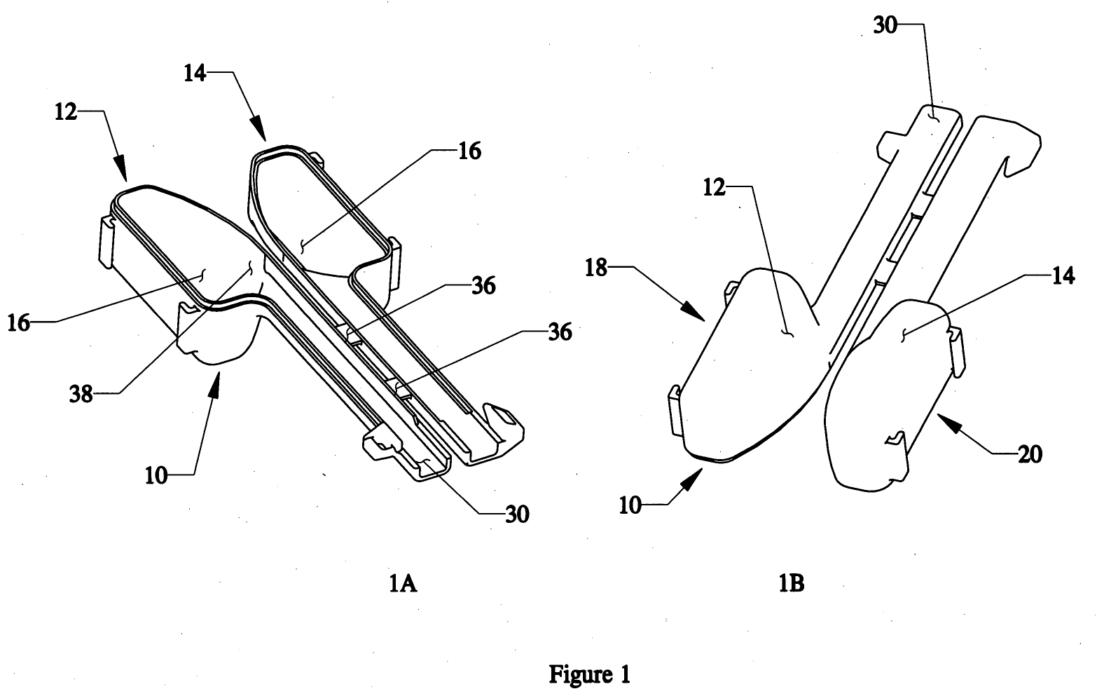

Now in reference to FIGS. 1A and 1B, one embodiment of the invention is described as the intra-oral sensor protection device 10. FIGS. 1A and 1B describe the protection device 10 in an open configuration, wherein the first sensor protection element 12 and second sensor protection element 14 is shown in an open configuration. Each sensor protection element has a fixed outer profile 18, 20. The outer profile can be provided in a number of shapes or contours, the outer profile having some relation and correspondence to the dictates of the size, shape and type of the sensor, such as the sensor size, model or brand of sensor, or if the sensor is wired or wireless, and the type of application or examination technique to be used.

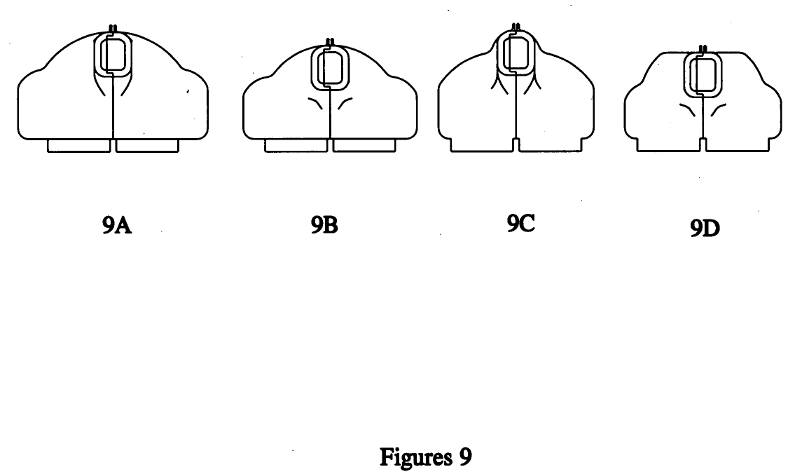

The sensor protection elements 12, 14 outer profile 18, 20 are fixed in that the contour and shape of the profile are retained in the use of materials and the manufacture thereof to serve and to provide a physical barrier between the patient and the sensor, particularly given the environment of intra-oral examination and a desire to retain a profile shape and contour to facilitate examination and repeated use, if desired. FIGS. 9A through 9D describe varying configurations of protection devices, sensor protection elements and fixed outer profiles as some alternative embodiments of the present invention.

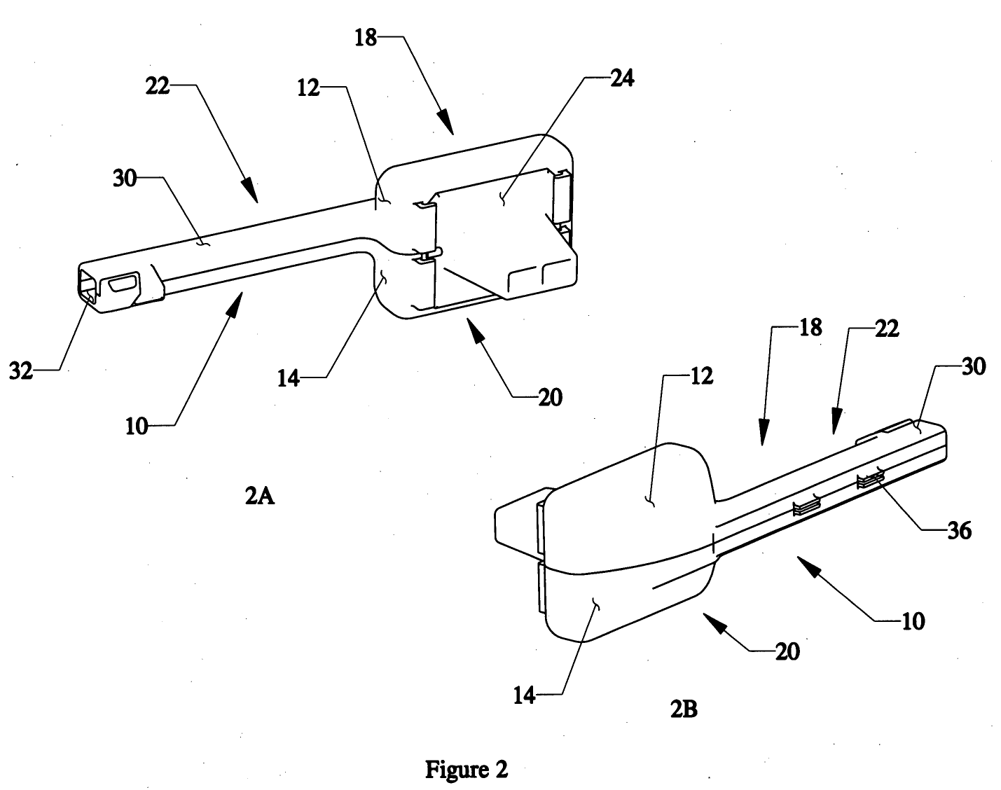

A sensor protection interior 16 is shown in FIG. 1A, an interior to each of the protection elements 12, 14, wherein the corresponding interior of FIG. 1B cannot be viewed given the orientation of the protection device 10. The interior is defined by the first and second sensor protection elements, and defined as a protection interior in a closed configuration of the protection elements (not shown in FIGS. 1A and 1B). FIGS. 2A and 2B as well as other disclosure herein describe a closed configuration of an intra-oral sensor protection device 10.

Now in reference to FIGS. 1 and 2, the first and second sensor protection elements 12, 14 may be retained in either the open or closed configurations, as described in some other embodiments described in the figures. The open configuration may correspond prior to the placement of the sensor within the intra-oral sensor protection device 10 or the removal of the sensor, such as after examination. The closed configuration may correspond to the sensor having been positioned within the sensor protection interior 16 and the first and second sensor protection elements having been releasably retained in the closed configuration in order to protect the sensor and the patient and to conduct examination.

As just described, the first and second sensor protection elements 12, 14 releasably retain open and closed configuration in some preferred embodiments. The protection elements will remain open, or alternatively remain closed, and further features of the present invention as described herein facilitate the retention of the open and closed configurations, such as connection and lock features as well as hinge elements and the materials thereof. As the protection elements are retained in either the open or closed configurations, the protection device 10 may be released to provide for the alternative configuration, and in some embodiments, to provide movement of the first and second protection elements in protecting the sensor and in conducting examination.

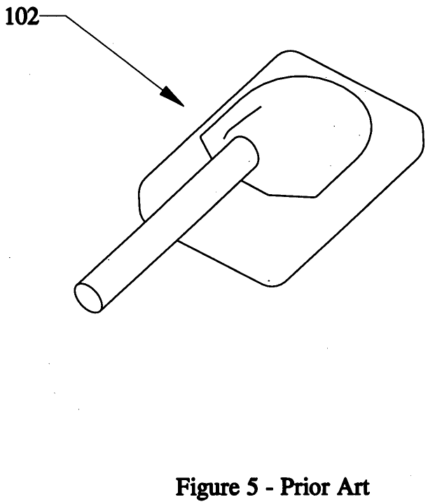

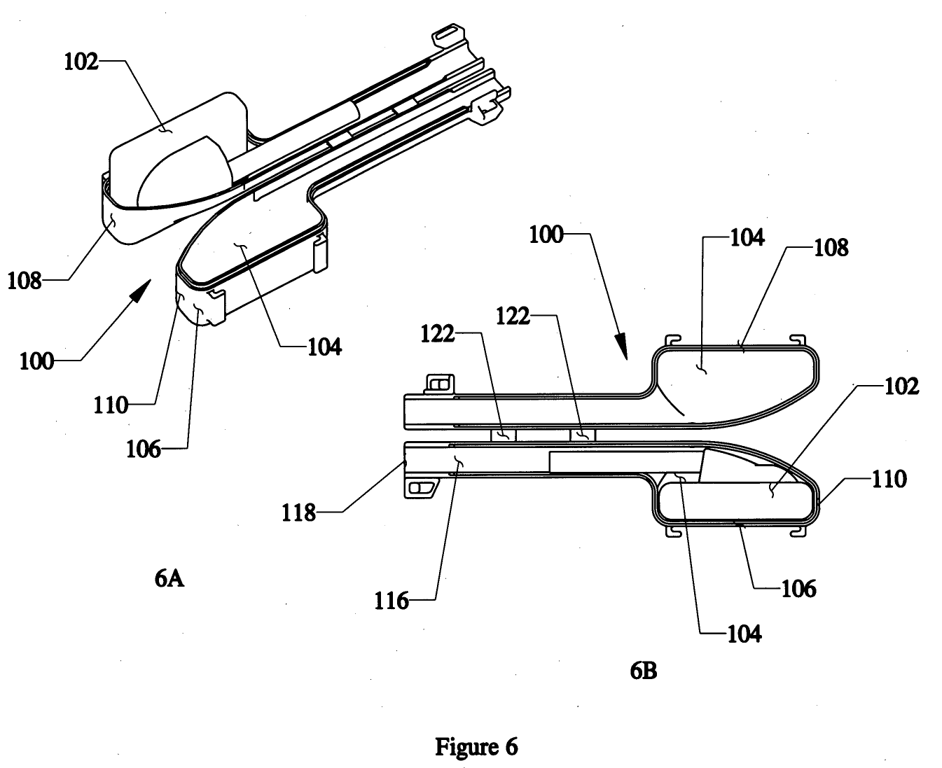

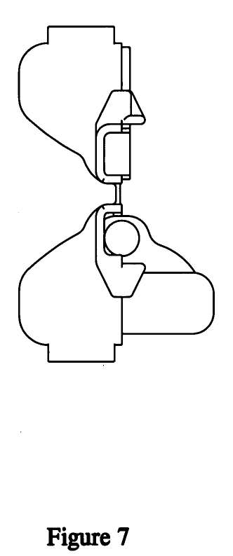

Accordingly, the first and second sensor protection elements in some preferred embodiments releasably retain a sensor that is positioned within the sensor protection interior 16. In the open configuration a sensor may be placed within the interior 16 and the first and second protection elements moved to a closed configuration (FIG. 2), thus defining the sensor protection interior and retaining a sensor placed within. Once configured in the closed configuration, the first and second sensor protection elements provide a fluid barrier about the extent of the sensor, as shown in FIGS. 6, 7 and 8 as description of intra-oral sensor protection device 100. FIG. 5 is a prior art description of a traditional sensor 102. Sensor 102 is positioned within the sensor protection interior 104 and protection elements 106, 108 are releasably retained from an open to a closed configuration such that the sensor 102 is releasably retained within the sensor protection interior 104 and the first and second sensor protection elements provide a fluid barrier about the extent of the sensor.

In still further embodiments, the intra-oral sensor protection devices 10, 100 as described in the figures may have a fixed profile sensor casing 22, 110 defined by the fixed outer profiles of the first and second sensor protection elements. The casing 22, 110 contain the extent of the sensor within the sensor protection interior and further provide a barrier about the extent of the sensor. The casing 22, 110 also facilitate positioning intra-orally the protection devices 10, 100 as described below.

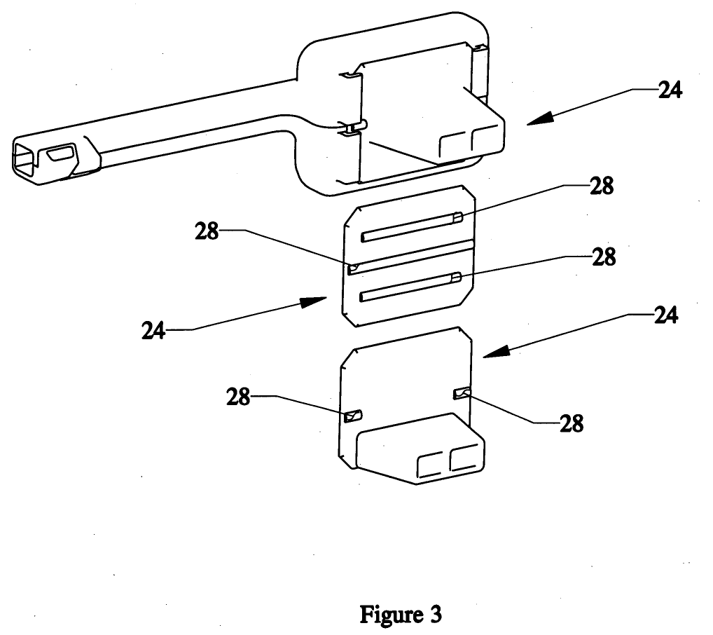

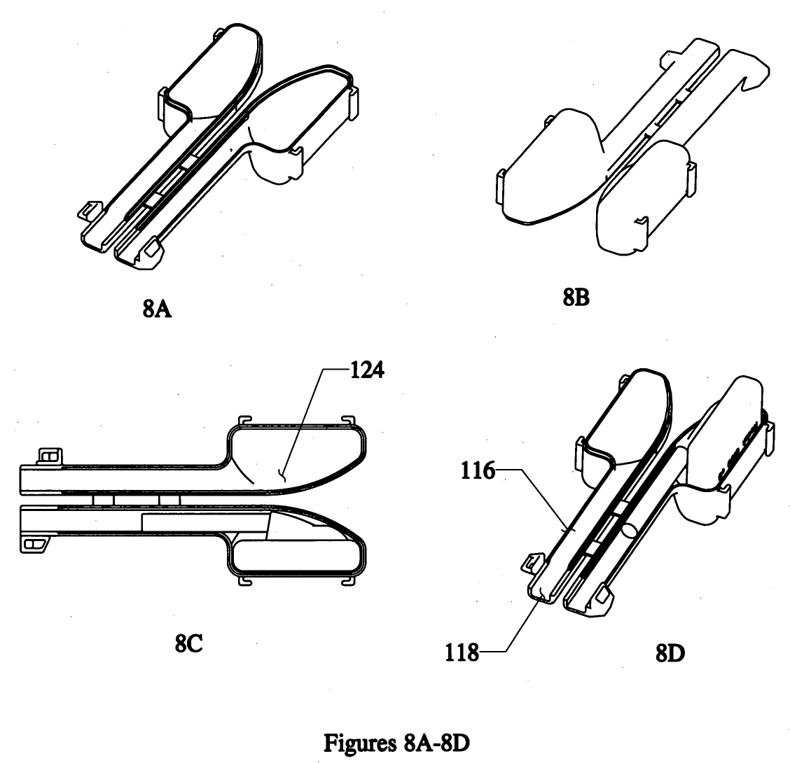

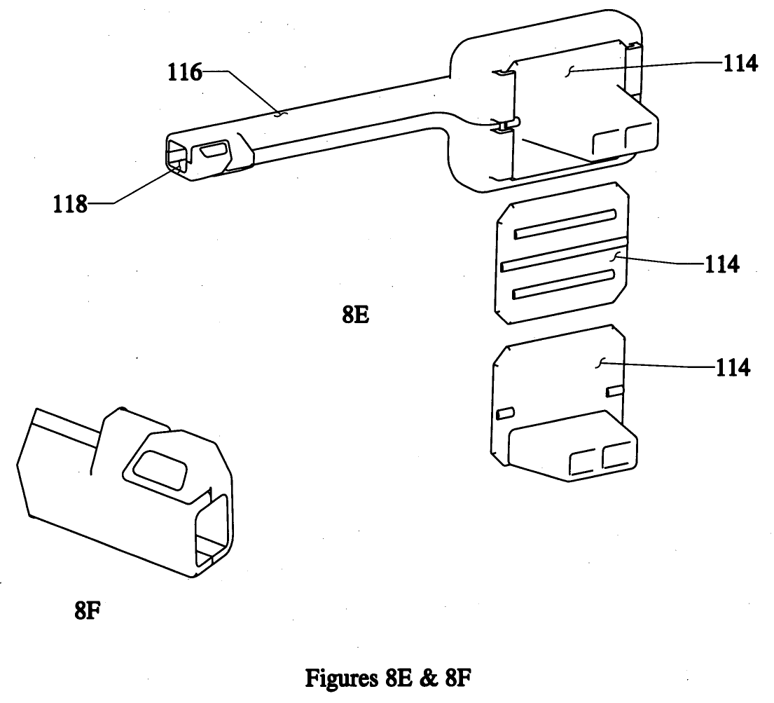

Now in reference to FIGS. 3 and 8E, the intra-oral sensor protection device may have a sensor casing positioning element 24, 114 of the sensor casing that in some embodiments may be connected with one of the first and second sensor protection elements. Independent of the features of the present invention, mechanical elements of the positioning element may be referred to as a bite tab or a bite wing. As described in the figures, the position element may be a featured connected with one of the sensor protection elements 12, 14 or may be provided as a unitary feature and element of the sensor protection element. FIG. 3 describes a sensor casing positioning element and its features in some embodiments. In preferred embodiments, such as those described in FIGS. 2, 3, 6, 7 and 8, the sensor casing positioning element is adjustably connected with one of the sensor protection elements. In some embodiments, and as shown in the Figures, the sensor casing positioning element 24, 114 may be adjusted such that configurations for bite wing, anterior and posterior of the sensor protection device may be accomplished. The sensor casing positioning element may in some preferred embodiments be connected with one of the sensor protection element and adjusted through the connection. In one embodiment, shown in FIG. 3, the positioning element 24 may be connected to the sensor protection element by sliding the positioning element within channels 26 up or down the sensor protection element. More broadly presented, guide elements such as the channels 26 shown in the figures are connected with the first and second sensor protection elements, the guide elements releasably retaining the first and second sensor protection elements in a closed configuration in combination with the sensor casing positioning element 24 in that the positioning element must be removed in order to open the sensor protection elements 12, 14.

The embodiments described in the Figures may provide the positioning element 24 connected to the sensor protection element by sliding the positioning element within channels on or off the sensor protection element, allowing for the positioning element to be removed or replaced. Alternatively, as shown in FIG. 3, the positioning element 24 may provide the releasable retainment of the sensor protection elements, wherein the positioning element when slid within the channels of the sensor protection elements releasably retains the sensor protection elements in a closed configuration. Alternatively, once removed, the protection elements may be released to an open configuration.

Embodiments of the invention can also provide positioning elements 24 with position element retention elements 28. In some embodiments, the position element retention elements 28 may be centering elements, and may be locating bumps preferably of non-slip material. FIG. 8E also describes alternative position element retention elements in light of an alternative embodiment of the intra-oral sensor protection device 100.

In still further embodiments, the invention may be embodied as an intra-oral sensor protection device having a sensor communication element 30, 116. Traditional digital sensors typically may have a wire or cable in data communication with the examination equipment in providing radiographical information. Accordingly, the present invention address concerns for protecting the sensor communication element while still affording fluid barrier and protection benefits by having sensor communication element 30, 116 contain a portion of the sensor communication of the sensor, and in some embodiments contain the wire or cable as a cable relief to reduce forces introduced on the wire and the sensor during examination or otherwise. In preferred embodiments, the sensor communication element 30, 116 is defined by the first and second sensor protection elements and may be integral of those elements. The sensor communication element 30, 116 has a communication port 32, 118 that affords the wire or cable to exit from the intra-oral sensor protection device in a manner that avoids unnecessary interference with traditional examination techniques, and particularly allows for the sensor and sensor protection device to be utilized better in an intra-oral application. The sensor communication element 30, 116 also allows the wire or cable to exit from the communication port 322, 118 such that the communication port is not subject to fluid entry, and further in light of the fluid barrier features described above.

The sensor communication element 30, 116 and the fluid barrier communication port 32, 118 may provide fluid barrier features consistent with the sensor protection elements by providing a fluid barrier about the extent of the sensor, and as further described below. As may be seen, for example, in FIG. 6, seal elements above and below the sensor communication are provided in the communication port 32, 118, such as at the nearest end of the sensor communication element 30, 116 to the sensor and as part of the sensor communication elements. In alternative embodiments, the sensor communication element may even be configured not as a means for entry or exit for a physical wire or cable, but may simply provide for wireless data transmission and wireless components of the sensor system to afford a wireless communication element. Without the need for an entry and exit port, the sensor communication elements may remain a solid component to afford fluid barrier and general barrier characteristics.

Again, in reference to the figures, an intra-oral sensor protection device can afford barrier protection, and in some embodiments, at least one or even both of the first and second sensor protection elements have a sensor protection connection element 36, 122. The sensor protection connection element in preferred embodiments would be a connection between the first and second sensor protection elements that may also serve to help provide the function of releasable retainment of the sensor protection elements, and even provides a fluid barrier to the sensor protection interior. In some preferred embodiments, a lap joint may be provided as the sensor protection connection element and serve to connect the first and second sensor protection elements. The mechanical joint is shown in the Figures; however, other connection means and barrier configurations may be utilized that would afford fluid barrier characteristics and that may also serve and provide the features described above. Barrier elements, such as the lap joint or joint barrier described in the figures, as well as hinge elements and sensor communication elements, such as cable protection elements, all serve to afford fluid protection and releasable retainment of the sensor protection elements and functionality of the intra-oral sensor protection device generally.

Some preferred embodiments provide features that further facilitate changing the open and closed configurations of the sensor protection elements and in some embodiments, one or more hinge elements 36, 122 may be connected with the first and second sensor protection elements. The first and second sensor protection elements are releasably retained by the hinge element in either an open configuration or a closed configuration, and the hinge elements 36, 122 being connected with each of the sensor protection elements, assists in essentially opening and closing the intra-oral sensor protection device, being connected with the sensor protection elements in providing hinge functionality.

Additionally, in some preferred embodiments, a sensor positioning element 24, 114 may be incorporated to adequately position the sensor within the intra-oral protection device. In preferred embodiments, the sensor positioning element is within the sensor protection interior, the positioning element positioning the sensor within the sensor protection interior. Furthermore, and in some embodiments, locating and support elements, and in some embodiments pins, may be provided in association with the first and second sensor protection elements. The support and locating elements may be provided on the top, bottom, or combination of top and bottom within the sensor protection interior, and in some embodiments such as those of the figures be the interior surface within the sensor protection interior corresponding to the fixed outer profile, such as an internal shoulder accommodating the shape and contour of the sensor.

Additionally, in some preferred embodiments, an intra-oral sensor protection device can be provided with a locking element within the sensor protection interior, the locking element releasably retaining the first and second sensor protection elements in a closed configuration. In some embodiments, ridges, bumps or similar fastener elements may be incorporated to lock the first and second sensor protection elements in a securely closed configuration while affording the opportunity to easily change to the open configuration. Alternatively, a locking element may be provided external to the sensor protection interior. In one embodiment, and as described in FIG. 8, and in particular FIG. 8F, a locking element having a first and second lock fit arrangement can be provided either connected with or as a unitary feature of other aspects of the invention. In the embodiment of FIG. 8, the locking element is provided with the sensor communication element.

An opening element such as a detent, ridge, depression, notch, slot or similar mechanical feature may be provided exterior on the first or second or both sensor protection elements, and preferably on the exterior of the fixed profile sensor casing. The opening element provides a convenient and easy release of the first and second sensor protection elements from a closed configuration to an open configuration by allowing the user to apply finger pressure to each sensor protection element to comfortably force open the intra-oral sensor protection device.

Another aspect of the invention may provide for the provision of locking features that may assist in the securing the closing of the enclosure. In some embodiments, these features may be referred to as interference features. In some particular embodiments, the locking elements may comprise corresponding location elements and locking elements. These features may not only provide for a removable lock for portions of the enclosure, but may also serve to provide a locating feature for examination media within the enclosure.

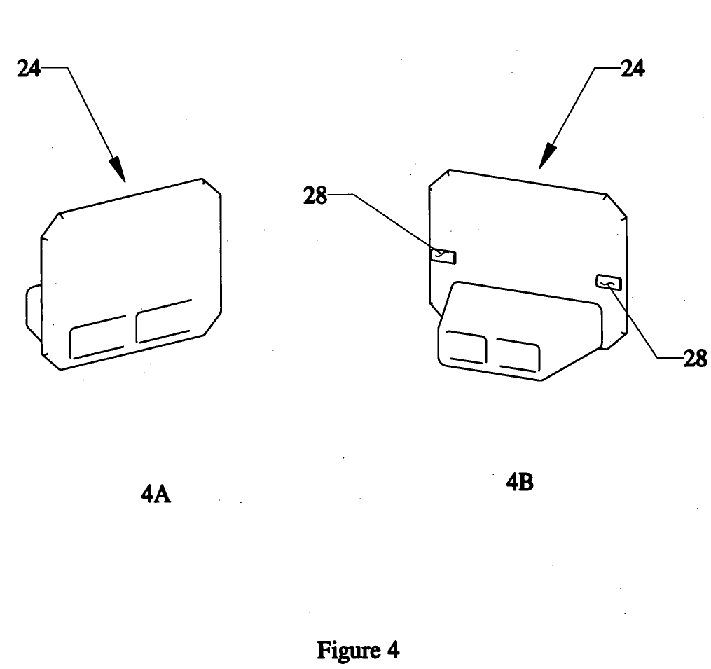

Again in reference to FIGS. 3 and 4, the enclosure may comprise bite wing or bite tab elements and further comprises locating features, and in some embodiments, locking features in association with the tab or wing. Accordingly, a bite tab or bitewing may be located and configured as a feature of the intra-oral sensor protection device and in reference to any examination technique. Some features may provide location and configuration of the tab or wing at different locations depending upon the intra-oral examination techniques used and may adjustably lock into place.

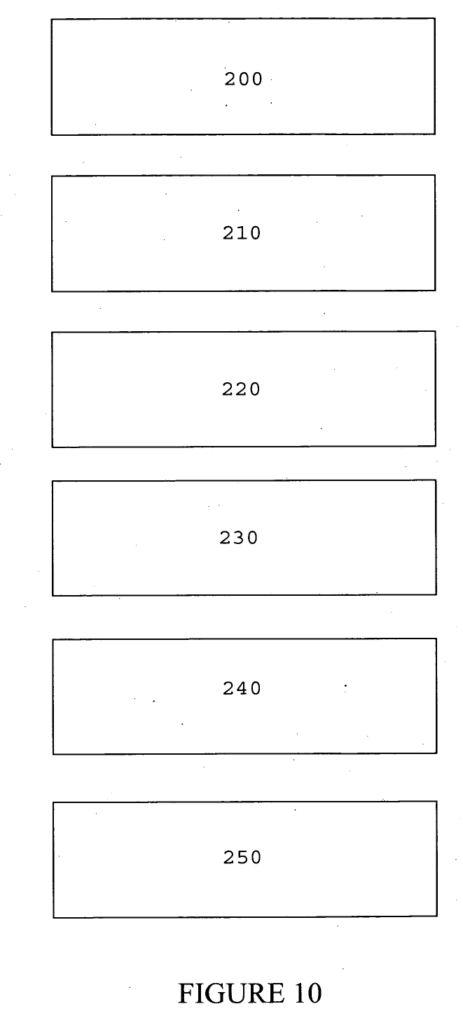

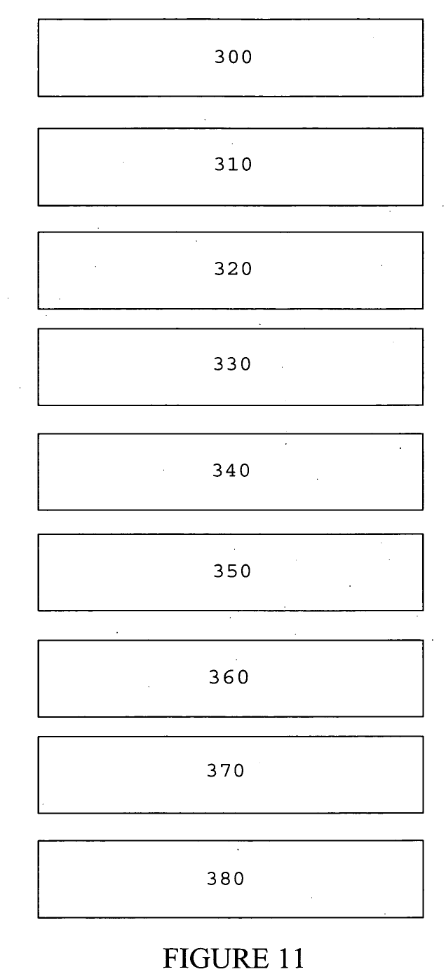

Additional embodiments of the invention comprise features that may be described in association with the known Kodak sensor of FIG. 5. FIGS. 10 and 11 describe the seal features, hinge features, open and closure of the intra-oral sensor protection device, and the sensor protection elements generally, in association with the Kodak sensor. A seal element, such as in some embodiments provided as a lap joint, comprise a fluid barrier of the intra-oral sensor protection device. The sensor may be provided within the enclosure and the seal element providing a fluid barrier once the enclosure is closed. Furthermore, and in some embodiments, a hinge is provided allowing for aspects of functionality of the present invention, including closing and opening of the enclosure. In some preferred embodiments, the hinge is an integral component of the enclosure, and may be provided as a molded one piece design in association with the enclosure. This type of hinge may be referred to as a living hinge in some embodiments.

Furthermore, and in reference to FIG. 10, present. invention may be embodied as a method of protecting a sensor during intra-oral examination. The features and functionality of the method and process embodiments correspond with the functionality previously described. Accordingly, in one embodiment, the method of protecting a sensor during intra-oral examination may begin with positioning a sensor in an interior of a first sensor protection element 200. The first sensor protection element may then be connected with a second sensor protection element 210 and the first and second sensor protection elements being releasably retained in a closed configuration 220. The sensor protection interior is then defined by the first and second sensor protection elements in the closed configuration 230, such that the sensor within the sensor protection interior may be releasably retained in said closed configuration 240, and accordingly a fluid barrier is provided with the first and second sensor protection elements about the extent of the sensor 250. Further, a barrier may be provided between the sensor and the patient with the first and second sensor protection. Again, retaining the sensor is important and the examination would be facilitated by positioning the sensor within said sensor protection interior.

In still a further method embodiment, the invention is a method of intra-oral examination. Now in reference to FIG. 11, a sensor for intra-oral examination is provided 300, preferably one intended for the particular examination technique. A sensor is positioned in an interior of a first sensor protection element 310 and the first sensor protection element is connected with a second sensor protection element 320. The first and second sensor protection elements are releasably retained in a closed configuration 330, such that a sensor protection interior is defined by the first and the second sensor protection elements in the closed configuration 340. The next step of this embodiment would be to releasably retain the sensor within the sensor protection interior in the closed configuration 350 to facilitate the examination, and further facilitated by providing a fluid barrier with the first and second sensor protection elements about the extent of the sensor 360. Then the sensor is placed in an intra-oral location of a patient for intra-oral examination 370 and the examination is performed on the patient 380.

Again, as previously described, a barrier may be provided between the sensor and the patient with the first and second sensor protection as a feature of the present invention and in utilizing the technology as a barrier. In the typical examination process, positioning a sensor casing with a positioning element connected with one of the first and second sensor protection elements can be crucial in obtaining a proper radiographed image. Positioning a sensor casing comprises adjustably positioning a sensor casing with an adjustable positioning element connected with one of the first and second sensor protection elements, and in preferred embodiments, positioning a bite wing or tab to accomplish the desired technique. In some embodiments, and as previously described a step may be taken to releasably retain the first and second sensor protection elements in a closed configuration with the sensor casing positioning element.

Further and additional steps may be performed, such as wherein positioning a sensor casing with a positioning element may be positioning a sensor casing in a bite wing configuration, or other known configurations, such as a posterior or anterior configuration, or even an occlusal configuration.

As can be easily understood from the foregoing, the basic concepts of the present invention may be embodied in a variety of ways. It involves one or more apparatus, device, processes, and methods of dentistry and intra-oral examination, and in some particular embodiments, to methods of business, and apparatus, devices, processes and methods directed to one or more patients, as well as the use of or incorporation of traditional technologies, as well as devices, apparatus, singular or plural, and assemblies to accomplish the appropriate function. In this application, the techniques are disclosed as part of the functionality of the various devices described and as steps that may be inherent to utilization. In addition, while some devices are disclosed, it should be understood that these not only accomplish certain methods but also can be varied in a number of ways. Importantly, as to all of the foregoing, all of these facets should be understood to be encompassed by this disclosure.

Any acts of law, statutes, regulations, or rules mentioned in this application for patent; or patents, publications, or other references mentioned in this application for patent are hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference.

Further, if or when used, the use of the transitional phrase “comprising” is used “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible.

We claim:

1. An intra-oral sensor protection device, comprising:
a first sensor protection element having a fixed outer profile;
a second sensor protection element having a fixed outer profile, wherein said first and said second protection elements may be releasably retained in either an open configuration or a closed configuration; and
a sensor protection interior defined by said first and said second sensor protection elements in said closed configuration;
wherein said first and second sensor protection elements releasably retain a sensor within said sensor protection interior in said closed configuration and wherein said first and second sensor protection elements provide a fluid barrier about the extent of said sensor.

2. An intra-oral sensor protection device as described in claim 1, further comprising a fixed profile sensor casing defined by said fixed outer profiles, said fixed profile sensor casing containing the extent of said sensor within said sensor protection interior and providing a barrier about the extent of said sensor.

3. An intra-oral sensor protection device as described in claim 1, wherein said first and second sensor protection elements define a sensor communication element to contain a portion of sensor communication of said sensor.

4. An intra-oral sensor protection device as described in claim 3, wherein said sensor communication element comprises a fluid barrier communication port.

5. An intra-oral sensor protection device as described in claim 4, wherein said sensor communication element comprises a wireless fluid barrier communication element.

6. An intra-oral sensor protection device as described in claim 4, wherein said sensor communication element comprises a fluid barrier cable relief.

7. An intra-oral sensor protection device as described in claim 1, wherein at least one of said first and second sensor protection elements comprise a sensor protection connection element.

8. An intra-oral sensor protection device as described in claim 7, wherein said sensor protection connection element comprises a connection releasably retaining said first and second sensor protection elements.

9. An intra-oral sensor protection device as described in claim 7, wherein said sensor protection connection element provides a fluid barrier to said sensor protection interior.

10. An intra-oral sensor protection device as described in claim 9, wherein said sensor protection connection element comprises at least one lap joint.

11. An intra-oral sensor protection device as described in claim 9, wherein said first and second sensor protection elements define a sensor communication element to contain a portion of sensor communication of said sensor and wherein said sensor protection connection element provides a fluid barrier to said sensor communication element.

12. An intra-oral sensor protection device as described in claim 1, further comprising a hinge element connected with said first and second sensor protection elements, wherein said first and second sensor protection elements may be releasably retained by said hinge element in either an open configuration or a closed configuration.

13. An intra-oral sensor protection device as described in claim 12, wherein said hinge element comprises a first and second hinge connected with said first and second sensor protection elements.

14. An intra-oral sensor protection device as described in claim 1, further comprising a sensor positioning element within said sensor protection interior, said positioning element positioning said sensor within said sensor protection interior.

15. An intra-oral sensor protection device as described in claim 2, further comprising a sensor casing positioning element connected with one of said first and second sensor protection elements.

16. An intra-oral sensor protection device as described in claim 15, wherein said sensor casing positioning element is adjustably connected with one of said first and second sensor protection elements.

17. An intra-oral sensor protection device as described in claim 16, wherein said sensor casing positioning element releasably retains said first and second sensor protection elements in a closed configuration.

18. An intra-oral sensor protection device as described in claim 17, further comprising guide elements connected with said first and second sensor protection elements, said guide elements releasably retaining said first and second sensor protection elements in a closed configuration in combination with said sensor casing positioning element.

19. An intra-oral sensor protection device as described in claim 1, further comprising a locking element within said sensor protection interior, said locking element releasably retaining said first and second sensor protection elements in a closed configuration.

20. An intra-oral sensor protection device as described in claim 2, further comprising an opening element on the exterior of said fixed profile sensor casing, said opening element providing release of said first and second sensor protection elements from a closed configuration to an open configuration.

21. A method of protecting a sensor during intra-oral examination, comprising the steps of:
positioning a sensor in an interior of a first sensor protection element;
connecting said first sensor protection element with a second sensor protection element;
releasably retaining said first and second sensor protection elements in a closed configuration;
defining a sensor protection interior by said first and said second sensor protection elements in said closed configuration;
releasably retaining said sensor within said sensor protection interior in said closed configuration; and
providing a fluid barrier with said first and second sensor protection elements about the extent of said sensor.

22. A method of protecting a sensor during intra-oral examination as described in claim 21, further comprising the step of providing a barrier between the sensor and the patient with said first and second sensor protection.

23. A method of protecting a sensor during intra-oral examination as described in claim 21, further comprising the step of positioning said sensor within said sensor protection interior.

24. A method of intra-oral examination, comprising the steps of:
providing a sensor for intra-oral examination;
positioning a sensor in an interior of a first sensor protection element;
connecting said first sensor protection element with a second sensor protection element;
releasably retaining said first and second sensor protection elements in a closed configuration;
defining a sensor protection interior by said first and said second sensor protection elements in said closed configuration;
releasably retaining said sensor within said sensor protection interior in said closed configuration;
providing a fluid barrier with said first and second sensor protection elements about the extent of said sensor;
placing said sensor in an intra-oral location of a patient for intra-oral examination; and
performing examination of the patient.

25. A method of intra-oral examination as described in claim 24, further comprising the step of providing a barrier between the sensor and the patient with said first and second sensor protection.

26. A method of intra-oral examination as described in claim 24, further comprising the step of positioning a sensor casing with a positioning element connected with one of said first and second sensor protection elements.

27. A method of intra-oral examination as described in claim 26, wherein said step of positioning a sensor casing comprises adjustably positioning a sensor casing with an adjustable positioning element connected with one of said first and second sensor protection elements.

28. A method. of intra-oral examination as described in claim 26, further comprising the step of releasably retaining said first and second sensor protection elements in a closed configuration with said sensor casing positioning element.

29. A method of intra-oral examination as described in claim 26, wherein said step of positioning a sensor casing with a positioning element comprises positioning a sensor casing in a bite wing configuration.

30. A method of intra-oral examination as described in claim 26, wherein said step of positioning a sensor casing with a positioning element comprises positioning a sensor casing in a posterior configuration.

31. A method of intra-oral examination as described in claim 26, wherein said step of positioning a sensor casing with a positioning element comprises positioning a sensor casing in an anterior configuration.

32. A method of intra-oral examination as described in claim 26, wherein said step of positioning a sensor casing with a positioning element comprises positioning a sensor casing in an occlusal configuration.
